(19) United States
(12) Patent Application Publication
(10) Pub. No.: US 2007/0055070 A1
(43) Pub. Date: Mar. 8, 2007

(54) Novel esters of lipoic acid

(76) Inventors: Lowell J. Lawrence, Lexington, KY (US); Rodney L. Eisenberg, Richmond, KY (US)

Correspondence Address: KING & SCHICKLI, PLLC, 247 NORTH BROADWAY, LEXINGTON, KY, 40507, US

(21) Appl. No.: 11/220,478

(22) Filed: Sep. 7, 2005

(51) Int. Cl. C07D 339/02

(52) U.S. Cl. 549/35

(57) ABSTRACT

A process is provided for producing lipoate esters from α-lipoic acid. The process comprises reacting α-lipoic acid with an alcohol and then adding a polymerization inhibitor such as L-cysteine.

### TECHNICAL FIELD

This invention generally relates to processes for the production of thioctic acid/lipoic acid esters.

### BACKGROUND OF THE INVENTION

Thioctic acid also known as α-lipoic acid, is well known in the art. Lipoic acid and its derivatives have a number of uses including but not limited to the treatment of liver disease, as an antidote to poisonous mushrooms, the treatment of diabetes, the treatment of asthma and as an antioxidant. Lipoic acid esters have uses as both prodrugs and as intermediates in the preparation of other lipoic acid derivatives.

It is well known that esters of lipoic acid may be prepared by heating the lipoic acid with an alcohol in the presence of an inorganic acid. Significantly, however, lipoic acid has a tendency to polymerize and such polymerization reduces the yield of the synthesis procedure. The present invention relates to a novel and improved process for producing lipoate esters from α-lipoic acid. Specifically, the α-lipoic acid and alcohol are reacted and then a polymerization inhibitor is added in order to reduce or eliminate the polymerization side reaction and increase lipoate ester yields.

### SUMMARY OF THE INVENTION

In accordance with the purposes of the present invention as described herein, a process is provided for producing lipoate esters from α-lipoic acid. The process comprises the step of reacting α-lipoic acid with an alcohol followed by the addition of a polymerization inhibitor. Next is the recovering of the lipoate ester product. More specifically, the process includes using L-cysteine or a derivative thereof as the polymerization inhibitor. The L-cysteine or the derivative thereof is added to the product mixture at a range of between about 0.5 and about 5.0 percent (weight/weight) with respect to the product lipoate ester.

The process further includes the step of performing the reacting step in the presence of an inorganic acid or acetyl chloride. In accordance with an additional aspect of the present invention where the alcohol of the reaction is C₇ or greater, the reacting step is performed in the presence of ethylchloroformate and triethylamine. Anhydrous THF may be used as a solvent for the α-lipoic acid in this reaction.

In accordance with yet another aspect of the present invention a compound is provided comprising

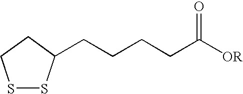

wherein R=—(CH₂)ₙCH₃ where n=6-31.

### DETAILED DESCRIPTION OF THE INVENTION

The present invention relates to a process for producing lipoate esters from α-lipoic acid including the pure R enantiomer, the pure S enantiomer as well as racemic and all other mixtures of enantiomers of α-lipoic acid. The process may be broadly described as comprising the step of reacting α-lipoic acid with an alcohol and then adding a polymerization inhibitor. L-cysteine or a derivative thereof (e.g. cysteine ethyl ester) is a particularly effective polymerization inhibitor for use in this process. More specifically, lipoic acid derivatives can reversibly polymerize by the action of a small amount of free thiol from reduced lipoic acid or other adventitious nucleophilic species acting on the disulfide bonds of the monomeric lipoic acid. L-cysteine provides a small quantity of free thiol to inhibit the polymerization reaction and to catalyze the corresponding depolymerization reaction. The L-cysteine is added in an amount of between about 0.5 and about 5.0% (weight/weight) of the product lipoate ester in order to be effective as a polymerization inhibitor.

For alcohols with C₁₋₆, the reacting step is performed in the presence of an inorganic acid. In one possible approach acetyl chloride is utilized to generate HCl from the reaction alcohol. The reaction may be written as illustrated by the following examples: 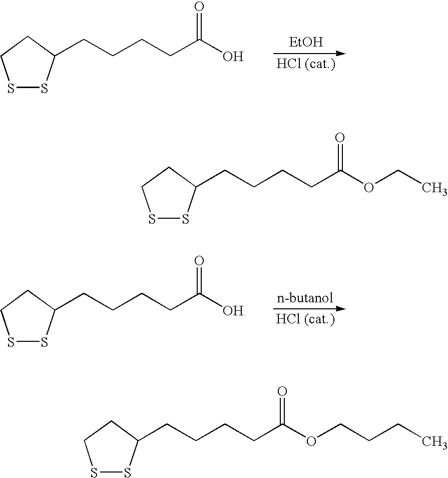
The reaction may be performed at room temperature or under heating.

For reaction with alcohols of C₇ or greater, the reacting step may be performed in the presence of ethyl chloroformate and triethylamine. Anhydrous THF may be used as a solvent for the α-lipoic acid in this approach. The reaction may be written as illustrated by the following examples:

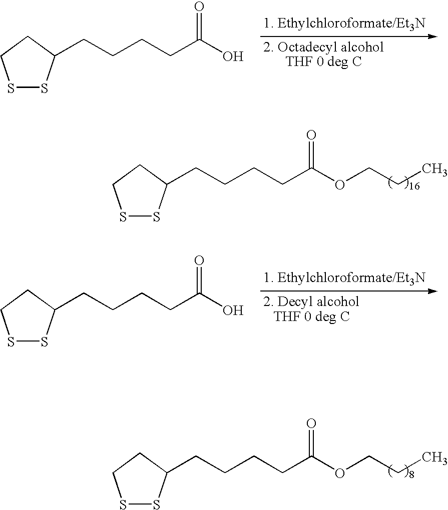

In yet another alternative embodiment, the α-lipoic acid is dissolved in a solvent (e.g. anhydrous dichloromethane (DCM)) and then reacted with dicyclohexylcarbodiimide (DCC) or analogous material such as N-(3-dimethylaminopropyl)-N¹-ethylcarbodiimide (EDC) and a C₇ or greater alcohol. This reaction is illustrated by the following examples:

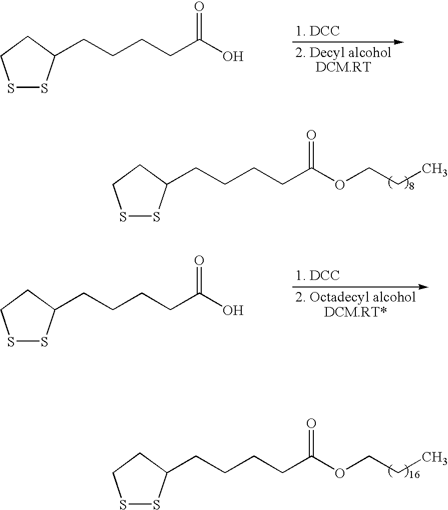

Useful compounds made in accordance with the teachings of the present invention include but are not limited to:

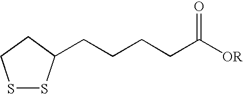

wherein R=—(CH₂)ₙCH₃ where n=1-31. This compound range includes the lower alkyl esters where R is methyl, ethyl, propyl, butyl, pentyl and hectyl and the higher alkyl esters where n=6-31.

The following synthesis and examples are presented to further illustrate the invention, but it is not to be considered as limited thereto.

### EXAMPLE 1

### Preparation of Lipoic Acid Ethyl Ester—100 g Scale

In a 2-L flask, was added absolute ethanol (1 L) followed by acetyl chloride (5.2 mL). The mixture was stirred for 2 hours. Lipoic acid (100.0 g) was added and stirred at room temperature overnight.

The reaction was quenched with the addition of solid sodium bicarbonate (25 g) and stirred for 4 h. The heterogeneous mixture was filtered through celite and L-cysteine (1 g) added. The solution was concentrated under reduced pressure while keeping the temperature less than 20° C. The resulting yellow oil was stored at ≦−5° C.

### EXAMPLE 2

### Preparation of Lipoic Acid Butyl Ester—100 g Scale

In a 2-L flask, was added n-butanol (1 L) followed by acetyl chloride (5.2 mL). The mixture was stirred for 2 hours. To the stirring mixture was added L-cysteine (1 g) and stirring continued for 20 min. Lipoic acid (100.0 g) was added and stirred at room temperature overnight.

The reaction was quenched with the addition of solid sodium bicarbonate (25 g) and stirred for 4 h. The heterogeneous mixture was filtered through celite and L-cysteine (1 g) added. The solution was concentrated under reduced pressure while keeping the temperature less than 20° C. The resulting yellow oil was stored at ≦−5° C.

### EXAMPLE 3

### Preparation of Lipoic Acid Octadecyl Ester—5 g Scale

Lipoic acid (5.0 g) was dissolved in anhydrous THF (100 mL). The stirring solution was cooled to 0° C. and triethylamine (3.7 mL) added. The cold reaction was stirred 10 min and ethyl chloroformate (2.6 mL) added slowly. The reaction was stirred at 0 C for 20 min then 1-octadecanol (6.9 g) was added. After stirring for 20 min the cooling bath was removed and the reaction stirred to room temperature overnight. A solution of 5% citric acid (75 mL) was added and the mixture stirred for 10 min then the two phases separated. The organic layer was washed with saturated sodium bicarbonate, brine and dried over sodium sulfate. After filtration of the solids, cysteine ethyl ester (100 mg) was added and the volatiles removed under reduced pressure at less than 20 C. The waxy solid was stored at ≦−5° C.

### EXAMPLE 4

### Preparation of Lipoic Acid Decyl Ester—5 g Scale

Lipoic acid (5.0 g) was dissolved in anhydrous THF (100 mL). The stirring solution was cooled to 0° C. and triethylamine (3.7 mL) added. The cold reaction was stirred 10 min and ethyl chloroformate (2.6 mL) added slowly. The reaction was stirred at 0 C for 20 min then 1-decanol (4.9 g) added. After stirring for 20 min the cooling bath was removed and the reaction stirred to room temperature overnight. A solution of 5% citric acid (75 mL) was added and the mixture stirred for 10 min then the two phases separated. The organic layer was washed with saturated sodium bicarbonate, brine and dried over sodium sulfate. After filtration of the solids, cysteine ethyl ester (100 mg) was added and the volatiles removed under reduced pressure at less than 20 C. The waxy solid was stored at ≦−5° C.

### EXAMPLE 5

### Preparation of Cysteine Ethyl Ester Used in Examples 3 and 4

Combine 2 mL absolute ethanol and 1 equivalent (0.05 g) cysteine ethyl ester hydrochloride. Stir until in solution then add 1 equivalent (0.015 g) sodium methoxide. Stir 20 min and filter through Celite.

### EXAMPLE 6

### Preparation of Lipoic Acid Decyl Ester, DCC Method—5 g Scale

Lipoic acid (5 g) was dissolved in anhydrous dichloromethane (DCM, 200 mL) and stirred at room temperature under an inert atmosphere.

Dicyclohexylcarbodiimide (DCC, 6.0 g) was dissolved in DCM (10 mL) and added to the lipoic acid solution and the resulting mixture stirred for 30 min. A solution of 1-decanol (4.2 g) was dissolved in DCM (10 mL) and added to the solution. The reaction was stirred overnight at room temperature. The reaction was filtered and washed three times with an aqueous solution consisting of 1M NaOH and 1M NaCl, dried over MgSO₄ and filtered. Cysteine ethyl ester (100 mg) was added and the volatiles evaporated under reduced pressure. The resulting solid was stored at less than −5° C.

### EXAMPLE 7

### Preparation of Lipoic Acid Decyl Ester, EDC Method—2 g Scale

Lipoic acid (2 g) was dissolved in DCM (75 mL) and the solution stirred at room temperature. Via syringe was added EDC and the reaction mixture stirred for 35 min. 2.04 g of 1-decanol was dissolved in DCM (5 mL) and the mixture stirred overnight. The mixture was washed with a solution of 0.5 M HCl, then saturated NaHCO₃ followed by a brine solution and dried with MgSO₄. After filtration of the solids 50 mg of ethyl cysteine was added and the volatiles removed under reduced pressure. The resulting solid was stored at less than −5° C.

### EXAMPLE 8

### Showing Polymerization Inhibition and Reversibility of Polymerizaton Using Cysteine and Cysteine Ethyl Ester

A solution of cysteine ethyl ester (1 g) (yellow oil) was prepared in ethanol (5 mL) and warmed overnight at 40° C. A rubbery solid resulted which was insoluble in ethanol, DCM or DMSO (dimethylsulfoxide). To this mixture was added 50 mg of L-cysteine ethyl ester and the solution heated several days with stirring in ethanol.

Depolymerization was observed as evidenced by the rubbery solution slowly dissolving in the ethanolic solution. A control sample with no cysteine added failed to redissolve under the same conditions. A similar sample of lipoic acid ethyl ester in ethanol containing L-cysteine ethyl ester remained in solution under the same conditions.

In summary, numerous benefits have been described which result from employing the concepts of the present invention. The utilization of L-cysteine after reaction of α-lipoic acid with an alcohol functions to inhibit polymerization of the α-lipoic acid and increases lipoate ester yields.

The invention has been described herein with reference to certain preferred embodiments. Obvious variations and modifications thereof will become apparent to those skilled in the art and, accordingly, the invention is not to be considered as being limited thereto.

What is claimed:

1. A process for producing lipoate esters from α-lipoic acid, comprising:
reacting α-lipoic acid with an alcohol to produce a lipoate ester product and then adding a polymerization inhibitor.

2. The process of claim 1, including using L-cysteine or a derivative thereof as said polymerization inhibitor.

3. The process of claim 2, including performing said reacting step in the presence of an inorganic acid.

4. The process of claim 2, including performing said reacting step in the presence of ethylchloroformate and triethylamine.

5. The process of claim 2, including performing said reacting step in the presence of acetyl chloride.

6. The process of claim 2, including using anhydrous THF as a solvent.

7. The process of claim 2 including recovering said lipoate ester product after adding said polymerization inhibitor.

8. A process for producing lipoate esters from α-lipoic acid, comprising:
reacting α-lipoic acid with a C₇-C₃₂ alcohol in the presence of ethylchloroformate and triethylamine to produce a lipoate ester product.

9. The process of claim 8 further including using anhydrous THF as a solvent.

10. The process of claim 8, including adding a polymerization inhibitor after producing said lipoate ester product.

11. The process of claim 10, including using L-cysteine or a derivative thereof as said polymerization inhibitor.

12. A process for producing lipoate esters from α-lipoic acid, comprising:
reacting α-lipoic acid with a C₇-C₃₂ alcohol in the presence of dicyclohexylcarbodiimide to produce a lipoate ester product.

13. The process of claim 12, including adding a polymerization inhibitor after producing said lipoate ester product.

14. The process of claim 13, including using L-cysteine or a derivative thereof as said polymerization inhibitor.

15. A process for producing lipoate esters from α-lipoic acid, comprising:
reacting α-lipoic acid with an alcohol in the presence of a polymerization inhibitor.

16. The process of claim 15, including using L-cysteine or a derivative thereof as said polymerization inhibitor.

17. A compound, comprising: 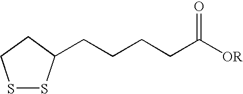
wherein R=—(CH₂)ₙCH₃ where n=6-31.
